(19) United States
(12) Patent Application Publication
(10) Pub. No.: US 2025/0108196 A1
(43) Pub. Date: Apr. 3, 2025

(54) PATCH WITH MICRONEEDLES AND METHODS OF USE

(71) Applicant: Boston Scientific Scimed, Inc., Maple Grove, MN (US)

(72) Inventors: Daragh Nolan, Youghal (IE); Robert Edward Lund, Saint Michael, MN (US); Sean Cooney, Meelick (IE); Brian P. Watschke, Minneapolis, MN (US); Thomas Sinnott, Enniscorthy (IE)

(21) Appl. No.: 18/894,898

(22) Filed: Sep. 24, 2024

Related U.S. Application Data
(60) Provisional application No. 63/586,537, filed on Sep. 29, 2023.

(51) Int. Cl. A61M 37/00

(52) U.S. Cl. CPC A61M 37/0015; A61M 2037/0023; A61M 2037/0061; A61M 2205/0244; A61M 2210/1089; A61M 2210/1475; A61M 2210/167; A61M 2210/168

(57) ABSTRACT

According to a general aspect, an apparatus includes a substrate, a plurality of microneedles, and a medicinal composition. The substrate has a surface. The plurality of microneedles is disposed on the surface of the substrate. The medicinal composition is configured to be delivered to a body of a person when the substrate is adhered to the body of the person. In some implementations, the medicinal composition is configured to help blood flow within bodily tissue of the person.

### CROSS-REFERENCE TO RELATED APPLICATION

This application claims priority to U.S. Provisional Patent Application No. 63/586,537, filed on Sep. 29, 2023, entitled “PATCH WITH MICRONEEDLES AND METHODS OF USE”, the disclosure of which is incorporated by reference herein in its entirety.

### TECHNICAL FIELD

This disclosure relates generally to transdermal drug delivery devices and, more specifically, to transdermal drug delivery devices configured to be used to help treat erectile dysfunction.

### BACKGROUND

One treatment for chronic, organic, male erectile dysfunction includes injecting medications via a syringe and needle into a penis of a person. These injections may be self-administered. In some cases, such injections may be painful and/or may cause scars to form. Additionally, there is a risk of infection at the injections site. Furthermore, bleeding or bruising may occur particularly if the injection is improperly administered, for example, if the needle contacts a blood vessel during the injection process.

### SUMMARY

According to a general aspect, an apparatus includes a substrate, a plurality of microneedles, and a medicinal composition. The substrate has a surface. The plurality of microneedles is disposed on the surface of the substrate. The medicinal composition is configured to be delivered to a body of a person when the substrate is adhered or coupled to the body of the person.

In some implementations, the apparatus includes an adhesive. In some implementations, the apparatus includes an adhesive disposed on the surface of the substrate.

In some implementations, the medicinal composition is disposed within at least one of the plurality of microneedles.

In some implementations, the plurality of microneedles is configured to dissolve when disposed within the body of the patient. In some implementations, the plurality of microneedles is formed of a bioabsorbable material.

In some implementations, the medicinal composition is in the form of a liquid. In some implementations, the medicinal composition is in the form of a gel. In some implementations, the medicinal composition is in the form of a solid.

In some implementations, the medicinal composition is configured to help increase blood flow within bodily tissue of the patient. In some implementations, the medicinal composition includes alprostadil.

In some implementations, the medicinal composition is a first medicinal composition, the apparatus further comprising a second medicinal composition. In some implementations, the medicinal composition is a first medicinal composition, the apparatus further comprising a second medicinal composition, the second medicinal composition being different than the first medicinal composition. In some implementations, the medicinal composition is a first medicinal composition, the apparatus further comprising a second medicinal composition, wherein the first medicinal composition is configured to help increase blood flow within the body of the patient and the second medicinal composition is configured to help prevent premature ejaculation.

In some implementations, the plurality of microneedles extends from the surface of the substrate a distance sufficient to penetrate an epidermis layer of the patient but insufficient to contact nerves of the person.

According to another aspect, a method includes providing patch having a substrate, a plurality of microneedles, and a medicinal composition and attaching the patch to a pelvic area of a body of a person.

In some implementations, the attaching includes attaching the patch to the pelvic area of the body of the person such that the plurality of microneedles contacts a perineum of the person, a penis of the person, a scrotum of the person, urethral tissue of the person, a vagina of a person, or other pelvic area of a person. In some implementations, the medicinal composition is configured to help increase blood flow within bodily tissue of the person. In some implementations, the attaching includes coupling the patch to a balloon catheter and inserting the patch and at least a portion of the balloon catheter into a urethra of the person.

In some implementations, the substrate and the plurality of microneedles are formed of bioabsorbable materials.

### BRIEF DESCRIPTION OF THE DRAWINGS

FIG. 1 schematically illustrates an apparatus according to an implementation.

FIG. 2 is a perspective view of an apparatus according to an embodiment.

FIG. 3 is a top view of the apparatus of FIG. 2.

FIG. 4 is a side view of the apparatus of FIG. 2.

FIG. 5 is a flow chart according to a method of an implementation.

FIG. 6 is a flow chart according to a method of an implementation.

FIG. 7 is a perspective view of a patch coupled to a balloon catheter according to an implementation.

FIG. 8A is a side view of a patch coupled to a balloon catheter in a deflated configuration.

FIG. 8B is an end view of the patch coupled to a balloon catheter of FIG. 8A in the deflated configuration.

FIG. 9A is a side view of a patch coupled to a balloon catheter in an expanded or inflated configuration.

FIG. 9B is an end view of the patch coupled to a balloon catheter of FIG. 9A in the expanded or inflated configuration.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices patches that include microneedles and a medicinal composition. The term patient may hereafter be used for a person who benefits from the medical device or the methods disclosed in the present disclosure. For example, the patient can be a person whose body is implanted using the medical device or benefits from the methods disclosed for operating the medical devices of the present disclosure. For example, in some embodiments, the patient may be a human male, a human female, or any other mammal.

The implementations discussed herein may include devices that provide treatment of erectile dysfunction. For example, such devices can be configured to be placed on or within a body of a person or patient.

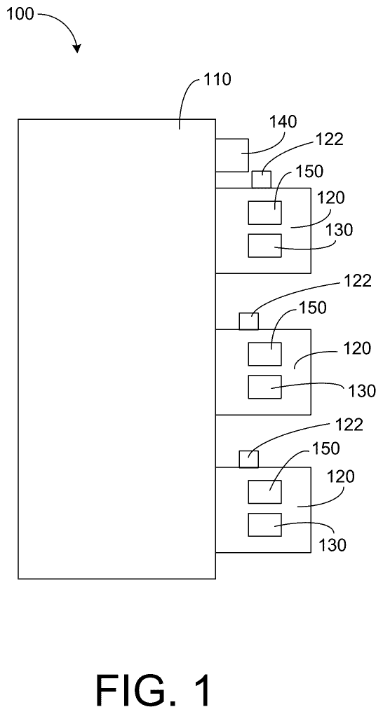

FIG. 1 schematically illustrates a device or apparatus 100 according to an implementation. The device 100 includes a substrate 110, a plurality of needles 120, and a medicinal composition 130. The device 100 may be placed on a portion of a body of a person or patient. The plurality of needles 120 are configured to penetrate an epidermis layer of the body of the person or patient. The medicinal composition 130 may then be delivered to the body of the person or patient via the plurality of needles 120.

The substrate 110 may be of any size or shape. In some implementations, the substrate 110 is a patch that may be placed directly on a surface of a body of the person or patient. For example, in some implementations, the substrate 110 may be placed on a perineum, a penis (such as a shaft of the penis), a scrotum, or a vagina of the person. In other implementations, the substrate 110 may be placed on the body of the person at a different location. In yet other implementations, the substrate 110 may be placed within a body of a person. For example, the substrate 110 may be placed within a urethra of a person, for example, via a balloon catheter or other device. In such implementations, the substrate 110 may be placed proximate to or coupled to urethral tissue.

In some implementations, the substrate 110 is formed of a biocompatible material. For example, the substrate 110 may be formed of a polymer or of silicon or of other materials. In some implementations, the substrate 110 is bioabsorbable and is configured to dissolve or biodegrade once it is placed within or on a body of a person.

In some implementations, the device 100 includes an adhesive or other material 140. The adhesive or other material 140 is configured to facilitate the adherence or coupling of the device 100, including the substrate 110 to the body of the person. In some implementations, the adhesive or other material 140 is located or disposed on a surface of the substrate 110.

The plurality of needles 120 extend from the substrate 110. For example, in some implementations, the plurality of needles 120 extend from a surface of the substrate 110. In some implementations, the needles 120 are configured to create small openings, channels, or pores in the skin of the person. In some implementations, the needles 120 are microstructures, such as microneedles. In some implementations, the needles or microneedles have a length (or otherwise extend from the substrate 110) such that they are long enough to penetrate an epidermis layer of the body of the person but not long enough to contact nerve or nerve endings of the person. In such implementations, the person may experience little or no pain when the needles 120 are used to penetrate the epidermis layer of the body of the person. In some implementations, the use of the microneedles reduces the risk of bruising or bleeding.

In some implementations, the needles 120 can be solid, hollow, coated, or hydrogel forming. The needles 120 can be configured to be disposed into or otherwise extend into the perineum, the penis, the scrotum, vagina, or urethral tissue of the person.

The needles 120 may house or contain the medicinal composition 130. The medicinal composition 130 may be in the form of a liquid, a gel, or a solid. In some implementations, the medicinal composition 130 is configured to help blood flow within bodily tissue of the person. In some implementations, the medicinal composition 130 includes alprostadil, papaverine, phentolamine and/or atropine. In other implementations, the medicinal composition 130 may include PDE-5s (Phosphodiesterase Inhibitors) such as Sildenafil, Tadalafil, Vardenafil, and/or Avanafil.

In some implementations, the needles 120 are configured to deliver the medicinal composition 130 immediately upon insertion into the body of the person. In other implementations, the needles 120 are configured to deliver the medical composition 130 over a period of time after insertion into the body of the person. In some cases, the delivery of the medicinal composition 130 over a period of time after insertion may help prevent priapism. In some implementations, the delivery of the medicinal composition 130 over a period of time may be achieved by incorporating the medicinal composition into the structure of the needle 120 and having the needle dissolve or degrade slowly.

In some implementations, the device 100 includes a second medicinal composition 150. The second medicinal composition may be configured to help prevent premature ejaculation. For example, the second medicinal composition 150 may include numbing agents such as lidocaine, benzocaine, and/or prilocaine. In other implementations, the second medicinal composition 150 may include Botox. In some implementations, the second medicinal composition may be disposed within or housed by the plurality of needles. In some implementations, the second medicinal composition 150 may be mixed with the first medicinal composition 130.

In some implementations, the needles 120 are configured to help facilitate the coupling of the device 100 to the body of the patient. For example, the needles 120 may include structures 122, such as coupling structures, that are configured to engage bodily tissue to help couple the device 100 to the body of the patient or person. In some implementations, the coupling structures 122 include projections, barbs, or other structures that extend from a surface of the needles 120.

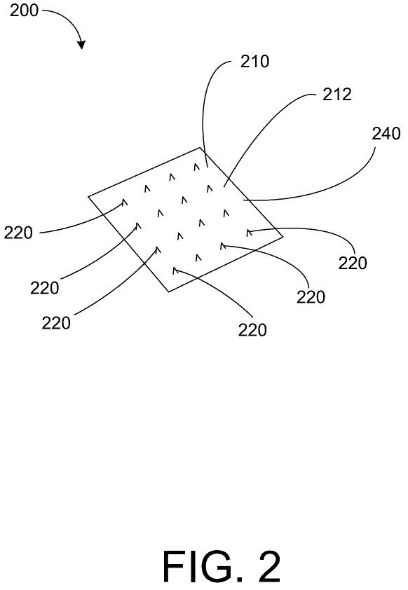

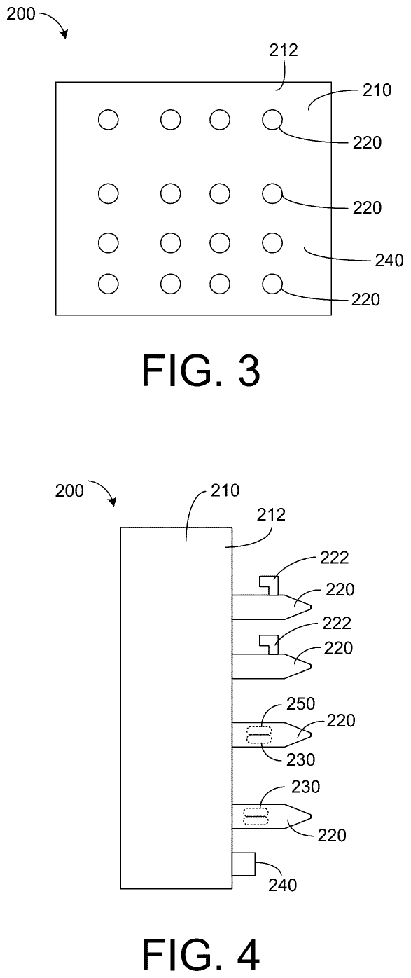

FIGS. 2-4 illustrate a device 200 according to an implementation. The device 200 includes a substrate 210, a plurality of needles 220, and a medicinal composition 230. The device 200 may be placed on a portion of a body of a person or patient. The plurality of needles 220 are configured to penetrate an epidermis layer of the body of the person or patient. The medicinal composition 230 may then be delivered to the body of the person or patient via the plurality of needles 220.

The substrate 210 may be of any size or shape. In some implementations, the substrate 210 is a patch that may be placed directly on a surface of a body of the person or patient. For example, in some implementations, the substrate 210 may be placed on a perineum, a penis (such as a shaft of the penis), a scrotum, or a vagina of the person. In other implementations, the substrate 210 may be placed on the body of the person at a different location. In yet other implementations, the substrate 210 may be placed within a body of a person. For example, the substrate 210 may be placed within a urethra of a person, for example, via a balloon catheter or other device. In such implementations, the substrate 210 may be placed proximate to or coupled to urethral tissue.

In some implementations, the substrate 210 is formed of a biocompatible material. For example, the substrate 210 may be formed of a polymer or of silicon or of other materials. In some implementations, the substrate 210 is configured to dissolve or biodegrade once it is placed within or on a body of a person.

In some implementations, the device 200 includes an adhesive or other material 240. The adhesive or other material 240 is configured to facilitate the coupling of the device 200, including the substrate 210 to the body of the person. In some implementations, the adhesive or other material 240 is located or disposed on a surface 212 of the substrate 210.

The plurality of needles 220 extend from the substrate 210. For example, in some implementations, the plurality of needles 220 extend from the surface 212 of the substrate 210. In some implementations, the needles 220 are configured to create small openings, channels, or pores in the skin of the person. In some implementations, the needles 220 are microstructures, such as microneedles. In some implementations, the needles or microneedles have a length (or otherwise extend from the substrate 210) such that they are long enough to penetrate an epidermis layer of the body of the person but not long enough to contact nerve or nerve endings of the person. In such implementations, the person may experience little or no pain when the needles 220 are used to penetrate the epidermis layer of the body of the person. In some implementations, the use of the microneedles 220 reduces the risk of bruising or bleeding.

In some implementations, the needles 220 can be solid, hollow, coated, or hydrogel forming. The needles 220 can be configured to be disposed into or otherwise extend into the perineum, the penis, the scrotum, a vagina or urethral tissue of the person.

The needles 220 may house or contain the medicinal composition 230. For example, the needles 220 may define a cavity that houses or contains the medicinal composition 230. In other implementations, the needles 220 may be formed with a material that is combined with the medicinal composition 230. The medicinal composition 230 may be in the form of a liquid, a gel, or a solid. In some implementations, the medicinal composition 230 is configured to help blood flow within bodily tissue of the person. In some implementations, the medicinal composition 230 includes alprostadil, papaverine, phentolamine and/or atropine. In other implementations, the medicinal composition 230 may include PDE-5s (Phosphodiesterase Inhibitors) such as Sildenafil, Tadalafil, Vardenafil, and/or Avanafil.

In some implementations, the needles 220 are configured to deliver the medicinal composition 230 immediately upon insertion into the body of the person. For example, the needles 220 may deliver the medicinal composition via capillary action. In other implementations, the needles 220 are configured to deliver the medical composition 230 over a period of time after insertion into the body of the person. In some cases, the delivery of the medicinal composition 230 over a period of time after insertion may help prevent priapism. In some implementations, the delivery of the medicinal composition 230 over a period of time may be achieved by incorporating the medicinal composition into the structure of the needle 220 and having the needle dissolve or degrade slowly.

In some implementations, the device 200 includes a second medicinal composition 250. The second medicinal composition may be configured to help prevent premature ejaculation. For example, the second medicinal composition 250 may include numbing agents such as lidocaine, benzocaine, and/or prilocaine. In other implementations, the second medicinal composition 250 may include Botox. In some implementations, the second medicinal composition may be disposed within or housed by the plurality of needles. In some implementations, the second medicinal composition 250 may be mixed with the first medicinal composition 230.

In some implementations, the needles 220 are configured to help facilitate the coupling of the device 200 to the body of the patient. For example, the needles 220 may include structures 222, such as coupling structures, that are configured to engage bodily tissue to help couple the device 200 to the body of the patient or person. In some implementations, the coupling structures 222 include projections, barbs, or other structures that extend from a surface of the needles 220. In some implementations, the needles 220 include both structures 222 and the medicinal compositions 230 and 250. In other words, a single needle may include both the structures 222 and the medicinal compositions 230 and 250.

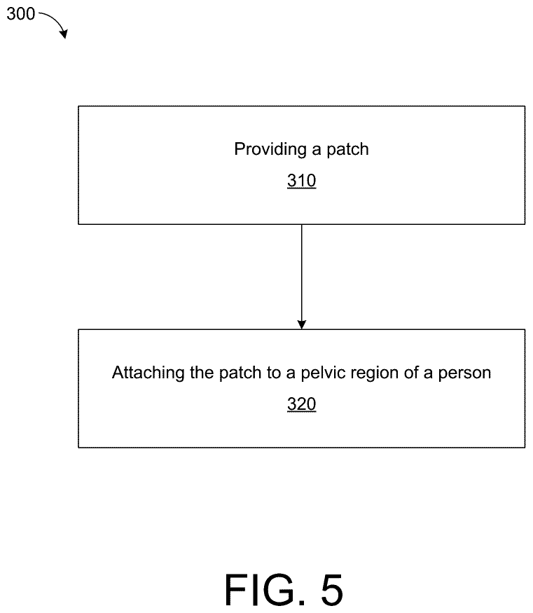

FIG. 5 is a flow chart of a method 300 according to an implementation. At step 310 a patch or device is provided. The patch or device may be similar to those described above such as device 100 or device 200. The device includes a substrate, a plurality of needles, and a medicinal composition. The device may be placed on a portion of a body of a person or patient. The plurality of needles is configured to penetrate an epidermis layer of the body of the person or patient. The medicinal composition may then be delivered to the body of the person or patient via the plurality of needles.

At step 320, the patch or device is attached to a portion of the body of the patient. For example, the patch or device may be attached to a pelvic region or area of the patient. In some implementations, the patch or device may be placed on a perineum, a penis (such as a shaft of the penis), a scrotum, or a vagina of the person. In other implementations, the substrate 210 may be placed on the body of the person at a different location.

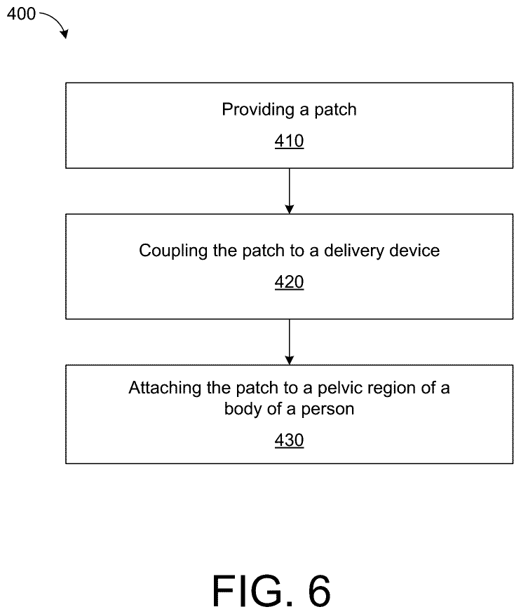

FIG. 6 is a flow chart of a method 400 according to an implementation. At step 410 a patch or device is provided. The patch or device may be similar to those described above such as device 100 or device 200. The device includes a substrate, a plurality of needles, and a medicinal composition. The device may be placed on a portion of a body of a person or patient. The plurality of needles is configured to penetrate a layer of the body of the person or patient. The medicinal composition may then be delivered to the body of the person or patient via the plurality of needles.

At step 420, the patch or device is coupled to a delivery device. The delivery device is configured to facilitate the placement of the patch or device. In some implementations, the delivery device is configured to facilitate the placement of the patch or device to a location within the body of the person or patient. For example, in some implementations, the delivery device is a catheter such as a balloon catheter. In such implementations, the patch or device may be folded or pleated with the balloon and may be inserted into the body of the patient in a deflated or low-profile configuration.

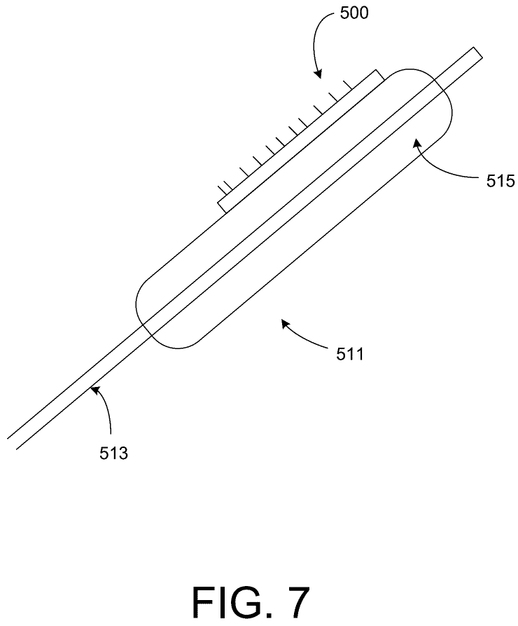

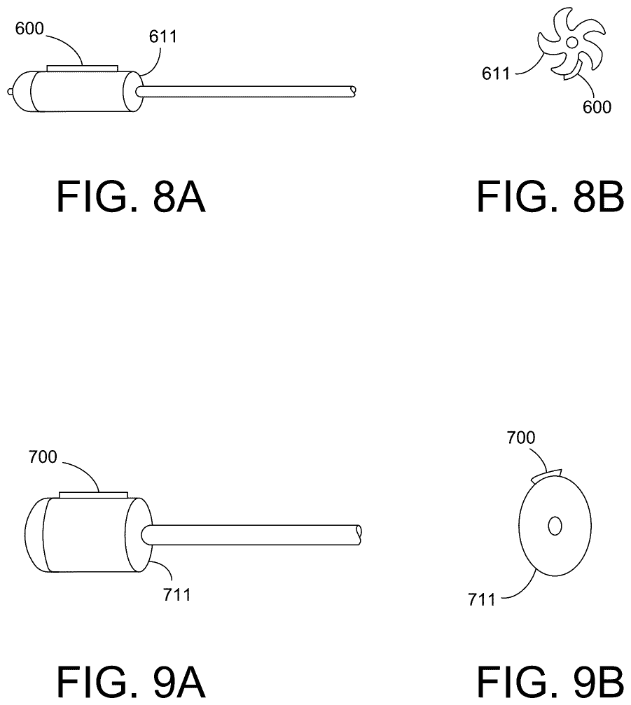

FIG. 7 is a perspective view of a patch 500 coupled to a balloon catheter 511 having a catheter 513 and a balloon or inflatable member 515 according to an implementation. FIG. 8A is a side view of a patch 600 coupled to a balloon catheter 611 in a deflated and folded or pleated configuration. FIG. 8B is an end view of the patch 600 coupled to the balloon catheter 611 in the deflated configuration. FIG. 9A is a side view of a patch 700 coupled to a balloon catheter 711 in an expanded or inflated configuration. FIG. 9B is an end view of the patch 700 coupled to the balloon catheter 711 the expanded or inflated configuration.

At step 430 of method 400, the patch or device is attached to a portion of the body of the patient using the delivery tool. For example, in some implantations, the delivery tool and the patch may be inserted into a portion of the body of the person or patient (for example at a pelvic or other region of the person or patient). For example, the patch and delivery tool may be inserted into a urethra of the patient. In some implementations, the delivery tool is a balloon catheter. In such cases, the balloon catheter may be inflated to attach the patch to a location within the urethra of the patient.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
a substrate having a surface;
a plurality of microneedles disposed on the surface of the substrate; and
a medicinal composition configured to be delivered to a body of a person when the substrate is adhered to the body of the person.

2. The apparatus of claim 1, further comprising an adhesive.

3. The apparatus of claim 1, further comprising an adhesive disposed on the surface of the substrate.

4. The apparatus of claim 1, wherein the medicinal composition is disposed within at least one of the plurality of microneedles.

5. The apparatus of claim 1, wherein the plurality of microneedles is configured to dissolve when disposed within the body of the person.

6. The apparatus of claim 1, wherein the plurality of microneedles is formed of a bioabsorbable material.

7. The apparatus of claim 1, wherein the medicinal composition is in the form of a liquid.

8. The apparatus of claim 1, wherein the medicinal composition is in the form of a gel.

9. The apparatus of claim 1, wherein the medicinal composition is in the form of a solid.

10. The apparatus of claim 1, wherein the medicinal composition is configured to help increase blood flow within bodily tissue of the person.

11. The apparatus of claim 1, wherein the medicinal composition includes alprostadil.

12. The apparatus of claim 1, wherein the medicinal composition is a first medicinal composition, the apparatus further comprising a second medicinal composition.

13. The apparatus of claim 1, wherein the medicinal composition is a first medicinal composition, the apparatus further comprising a second medicinal composition, the second medicinal composition being different than the first medicinal composition.

14. The apparatus of claim 1, wherein the medicinal composition is a first medicinal composition, the apparatus further comprising a second medicinal composition, wherein the first medicinal composition is configured to help increase blood flow within the body of the person and the second medicinal composition is configured to help prevent premature ejaculation.

15. The apparatus of claim 1, wherein the plurality of microneedles extends from the surface of the substrate a distance sufficient to penetrate an epidermis layer of the person but insufficient to contact nerves of the person.

16. A method, comprising:
providing a patch having a substrate, a plurality of microneedles, and a medicinal composition; and
attaching the patch to a pelvic region of a body of a person.

17. The method of claim 16, wherein the attaching includes attaching the patch to the pelvic region of the body of the person such that the plurality of microneedles contacts a perineum of the person, a penis of the person, a scrotum of the person, a vagina of the person, or urethral tissue of the person.

18. The method of claim 16, wherein the medicinal composition is configured to help increase blood flow within bodily tissue of the person.

19. The method of claim 16, wherein the attaching includes coupling the patch to a balloon catheter and inserting the patch and at least a portion of the balloon catheter into a urethra of the person.

20. The method of claim 16, wherein the substrate and the plurality of microneedles are formed of bioabsorbable materials.
